(19) United States
(12) Patent Application Publication
(10) Pub. No.: US 2026/0102076 A1
(43) Pub. Date: Apr. 16, 2026

(54) AUTOMATED BREATH ANALYSIS APPARATUS AND METHOD

(71) Applicant: BREATHONIX PTE. LTD., Singapore (SG)

(72) Inventors: Zhunan JIA, Singapore (SG); Fang DU, Singapore (SG)

(21) Appl. No.: 19/115,040

(22) PCT Filed: Sep. 26, 2023
(86) PCT No.: PCT/SG2023/050643

§ 371 Date: Mar. 25, 2025

(30) Foreign Application Priority Data
Sep. 28, 2022 (SG) 10202251197V

(51) Int. Cl. A61B 5/08; A61B 5/00; A61B 5/097; A61L 2/04; A61L 9/16; A61L 103/15

(52) U.S. Cl. CPC A61B 5/082; A61B 5/0075; A61B 5/0803; A61B 5/097; A61B 5/6887; A61B 5/7475; A61B 5/742; A61L 2/04; A61L 9/16; A61L 2103/15; A61L 2209/10

(57) ABSTRACT

The present disclosure generally relates to an automated breath analysis apparatus (100) and method (300). The apparatus (100) comprises: a set of input devices (110,112) for receiving identification data of a user; a sampling device (120) for receiving a breath sample from the user, the sampling device (120) comprising a nozzle (122) for the user to exhale the breath sample into the sampling device (120), the nozzle (122) arranged to hold a hygiene mouthpiece such that the user exhales the breath sample via the hygiene mouthpiece without touching the nozzle (122); and an analysis device (200) for receiving the breath sample from the sampling device (120) and for analysing the breath sample. The analysis device (200) comprises: a mass spectrometer (220) for measuring a number of biomarkers in the breath sample; and a computer device (230) for receiving the user identification data from the input devices (110,112) and for generating breath analysis results for the user based on the biomarker measurements.

### CROSS REFERENCE TO RELATED APPLICATIONS

The present disclosure claims the benefit of Singapore Patent Application 10202251197V filed on 28 Sep. 2022, which is incorporated in its entirety by reference herein.

### TECHNICAL FIELD

The present disclosure generally relates to an automated breath analysis apparatus and method. More particularly, the present disclosure describes various embodiments of an apparatus, such as a kiosk, and a method for performing automated breath analysis for people.

### BACKGROUND

Breath analysis is an emerging technology where the analysis of components present in exhaled human breath allows for monitoring of human health. An individual typically needs to visit a healthcare facility and the complex processes in breath analysis often require healthcare professionals to perform these processes and to operate the breath analysis equipment. Such conventional processes are labour intensive and have high operational costs.

Therefore, in order to address or alleviate at least one of the aforementioned problems and/or disadvantages, there is a need to provide an improved breath analysis apparatus and method.

### SUMMARY

According to a first aspect of the present disclosure, there is an automated breath analysis apparatus comprising: a set of input devices for receiving identification data of a user; a sampling device for receiving a breath sample from the user, the sampling device comprising a nozzle for the user to exhale the breath sample into the sampling device, the nozzle arranged to hold a hygiene mouthpiece such that the user exhales the breath sample via the hygiene mouthpiece without touching the nozzle; and an analysis device for receiving the breath sample from the sampling device and for analysing the breath sample. The analysis device comprises: a mass spectrometer for measuring a number of biomarkers in the breath sample; and a computer device for receiving the user identification data from the input devices and for generating breath analysis results for the user based on the biomarker measurements.

According to a second aspect of the present disclosure, there is an automated breath analysis method comprising: receiving, using a set of input devices, identification data of a user; receiving, using a sampling device, a breath sample from the user, the user exhaling the breath sample into a nozzle of the sampling device via a hygiene mouthpiece held on the nozzle and without touching the nozzle; communicating the breath sample from the sampling device to an analysis device for analysing the breath sample; measuring, using a mass spectrometer of the analysis device, a number of biomarkers in the breath sample; receiving, using a computer device of the analysis device communicative with the input devices and the mass spectrometer, the identification data of the user from the input devices; and generating, using the computer device, breath analysis results for the user based on the biomarker measurements.

An automated breath analysis apparatus and method according to the present disclosure are thus disclosed herein. Various features and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIGS. 1A to 1E are illustrations of an exterior of an automated breath analysis apparatus, according to embodiments of the present disclosure.

FIGS. 2A and 2B are illustrations of an interior of the automated breath analysis apparatus, according to embodiments of the present disclosure.

FIG. 3 is a flowchart illustration of an automated breath analysis method, according to embodiments of the present disclosure.

### DETAILED DESCRIPTION

For purposes of brevity and clarity, descriptions of embodiments of the present disclosure are directed to an automated breath analysis apparatus and method, in accordance with the drawings. While parts of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of features of particular embodiments. In a number of instances, well-known systems, methods, procedures, and components have not been described in detail so as to not unnecessarily obscure features of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment. As used herein, the terms “a” and “an” are defined as one or more than one. The use of “/” in a figure or associated text is understood to mean “and/or” unless otherwise indicated. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions. The terms “first”, “second”, etc. are used merely as labels or identifiers and are not intended to impose numerical requirements on their associated terms.

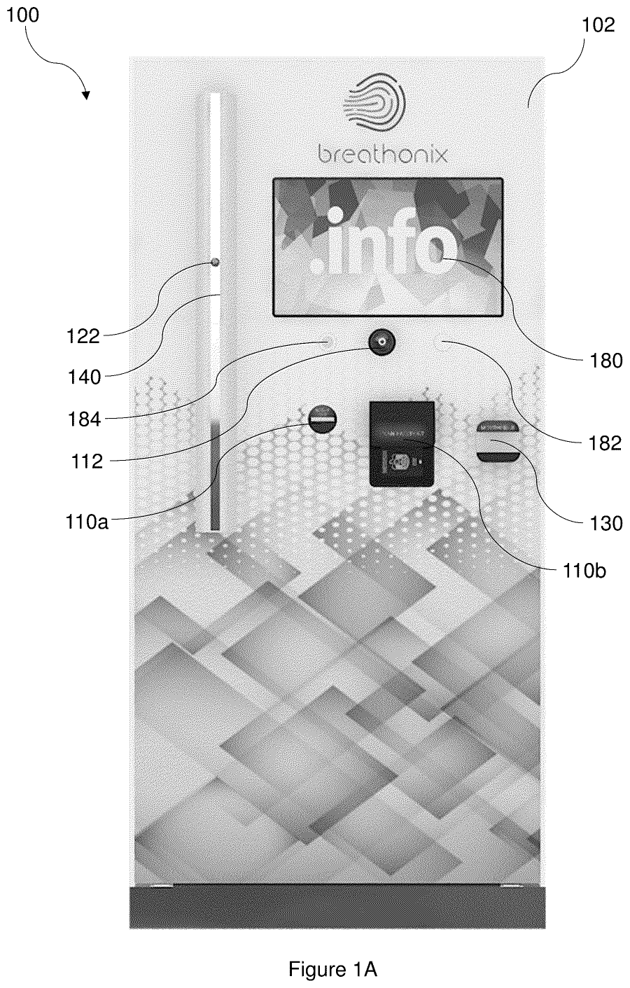

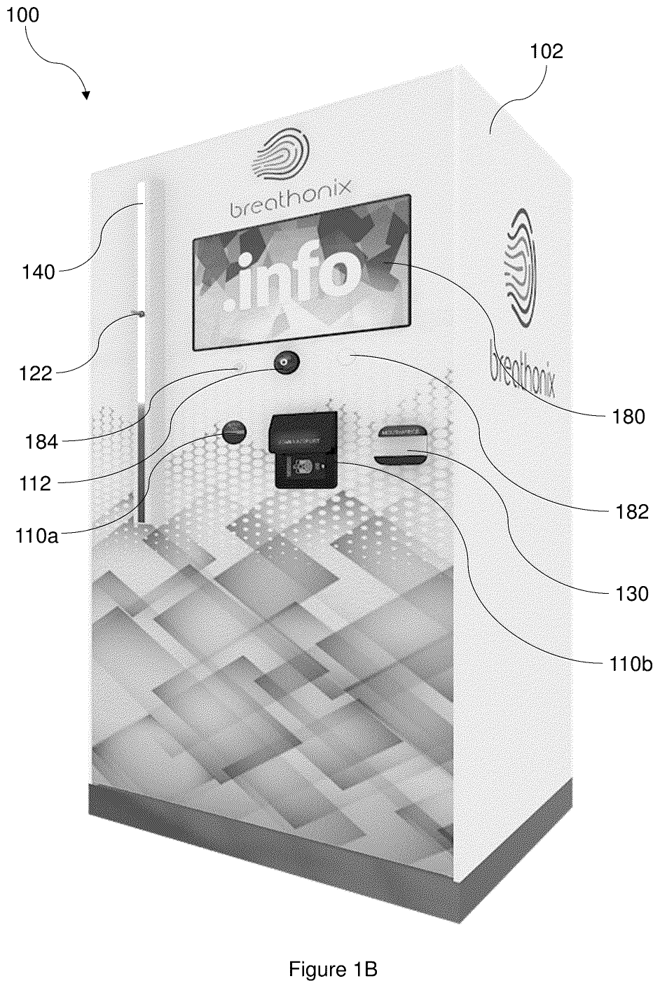

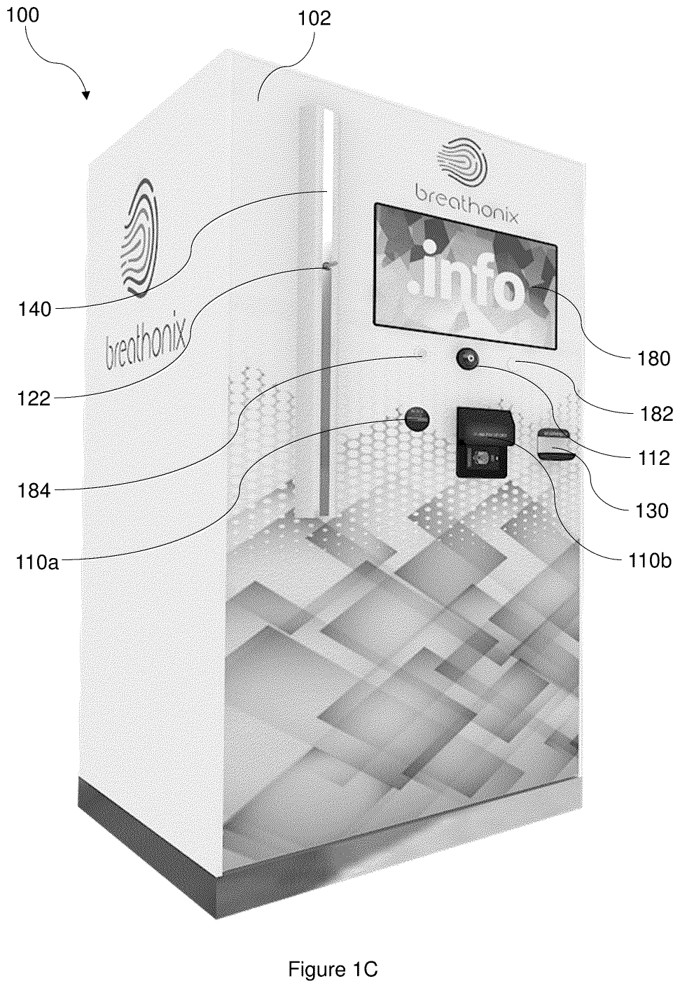

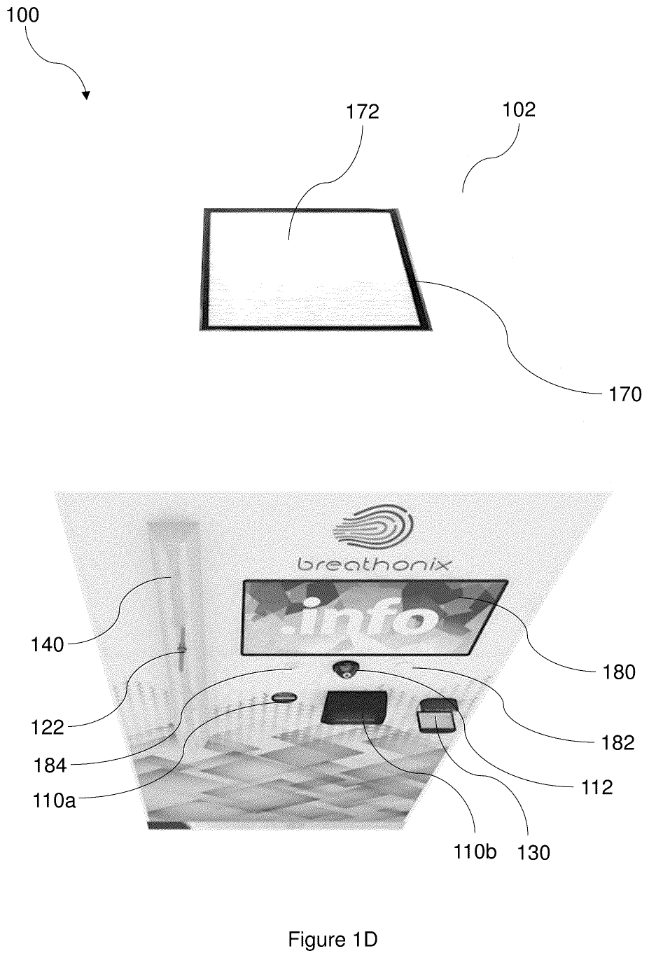

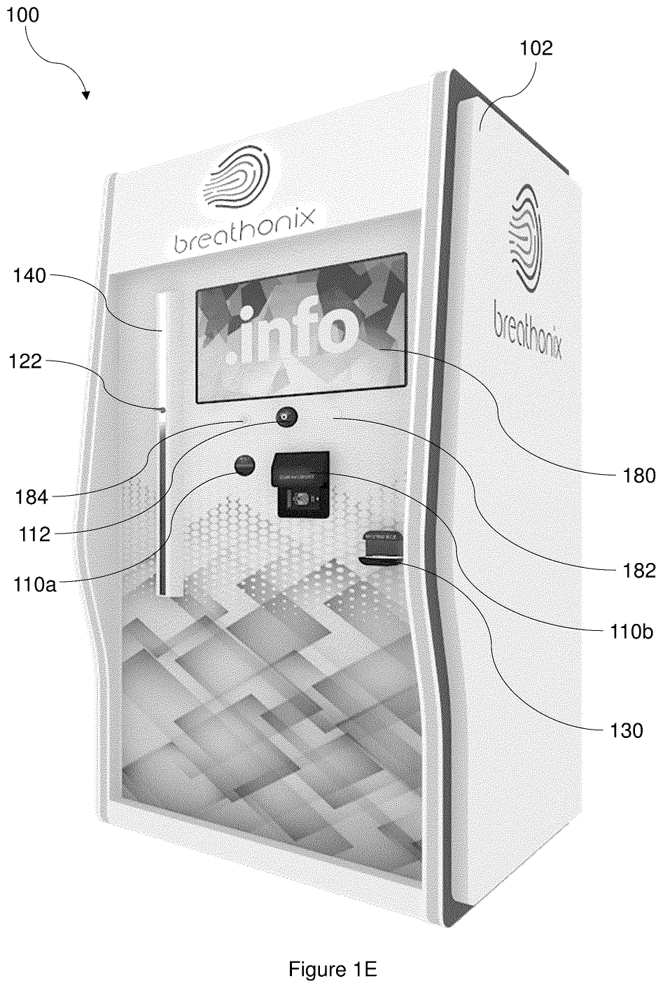

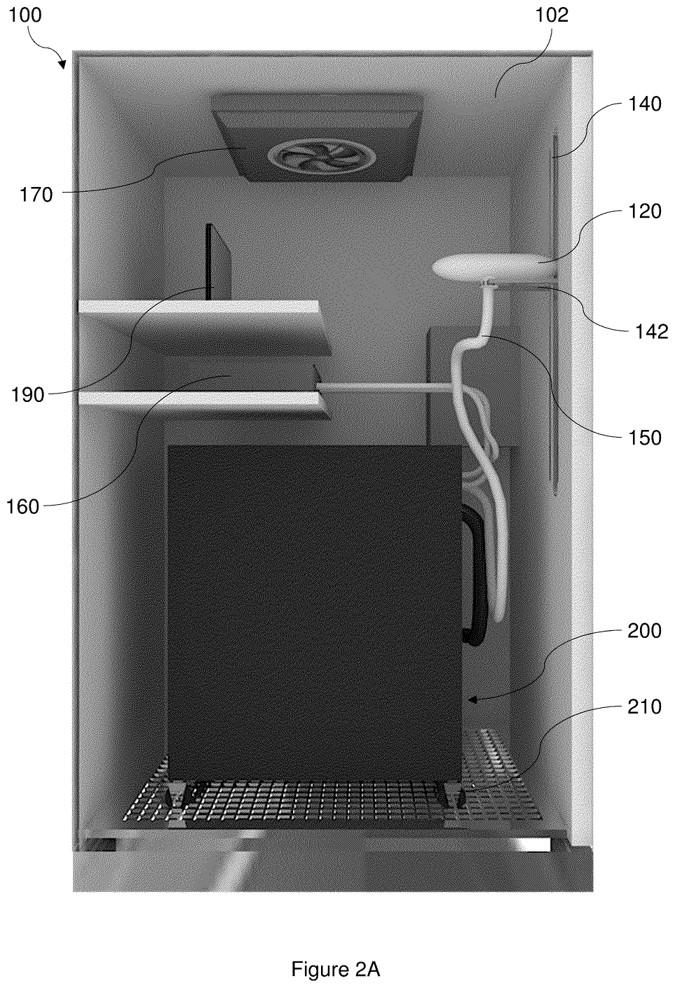

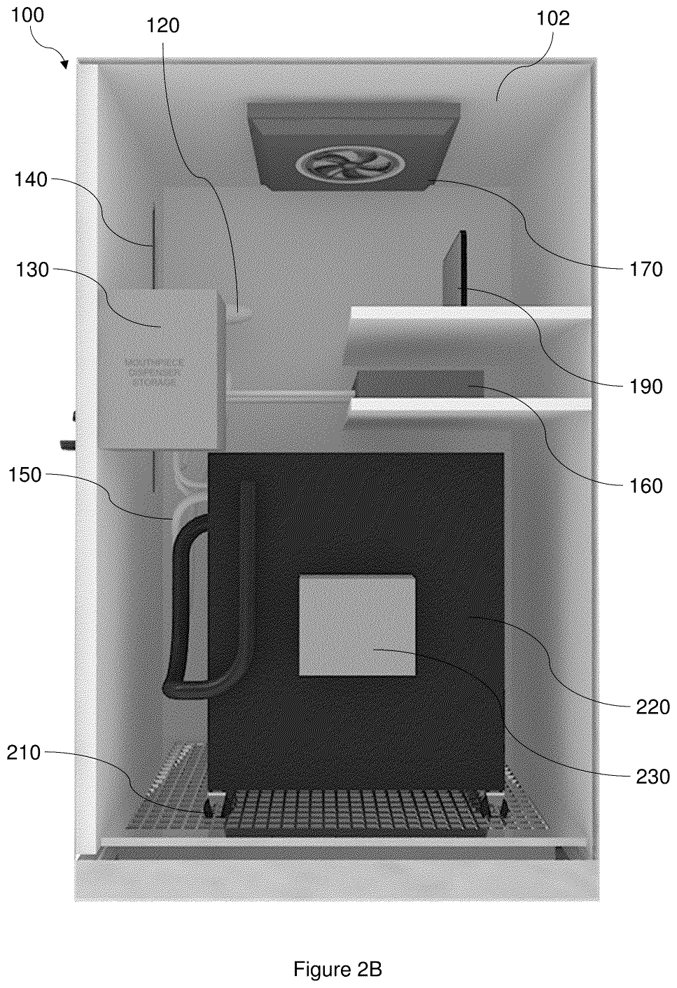

Representative or exemplary embodiments of the present disclosure describe an automated breath analysis apparatus 100. FIGS. 1A to 1D show one exemplary exterior design of the apparatus 100. FIG. 1E shows another exemplary exterior design of the apparatus 100. FIGS. 2A and 2B show an exemplary interior design of the apparatus 100. The apparatus 100 may be in the form of a kiosk similar to a vending machine and preferably located in public areas where users may pass by and use the apparatus 100 as necessary. More particularly, the apparatus 100 may include a housing body 102 which houses various components for performing the breath analysis, as described further below.

The apparatus 100 may be used by any user, subject, patient, host, or individual. The terms “user”, “subject”, “patient”, “host”, and “individual” may be used interchangeably herein.

The apparatus 100 includes a set of input devices for receiving identification data of the user. In many embodiments, the input devices include one or more scanning devices 110 for scanning an identification document of the user to thereby receive the user identification data. For example, the input devices include a first scanning device 110a for scanning an identification card of the user and/or a second scanning device 110b for scanning a passport of the user. Alternatively or additionally, the input devices include one or more imaging devices 112 for capturing image data of the user. For example, the imaging devices 112 include cameras that capture facial data of the user, wherein the user identification data includes the facial data contained in the image data of the user.

The apparatus 100 includes a sampling device 120 for receiving a breath sample from the user. The sampling device 120 includes a nozzle 122 for the user to exhale the breath sample into the sampling device 120. For example, the sampling device 120 is disposed inside the housing body 102 and the nozzle 122 protrudes outside the housing body 102. The nozzle 122 is arranged to hold a hygiene mouthpiece, such as a disposable one, such that the user exhales the breath sample via the hygiene mouthpiece without touching the nozzle 122. More specifically, the user places the hygiene mouthpiece over the nozzle 122 and positions his/her mouth over the hygiene mouthpiece and the nozzle 122, such that the mouth does not physically contact the nozzle 122 directly when exhaling the breath sample into the nozzle 122. The use of the hygiene mouthpiece prevents contamination of the nozzle 122 by the user and allows the next user to use the apparatus 100 safely.

In some embodiments, the apparatus 100 includes a dispenser 130 for storing a plurality of hygiene mouthpieces and for dispensing the hygiene mouthpiece to the user. The dispenser 130 may further be configured to dispense the hygiene mouthpiece in response to the input devices receiving the user identification data. For example, after the user scans his/her identification document using a scanning device 110, the dispenser 130 automatically dispenses a hygiene mouthpiece for the user to use. The hygiene mouthpieces are preferably individually packaged to avoid contamination and the user would need to remove the hygiene mouthpiece from the packaging and place it on the nozzle 122. The hygiene mouthpiece may include a one-way valve for the breath sample to flow from the mouth into the nozzle 122 and prevents any fluid flow from the nozzle 122 to the mouth. This automatic dispensation allows the user to perform the breath analysis himself/herself without needing an operator to issue the hygiene mouthpiece. The dispenser 130 would be manually refilled with fresh hygiene mouthpieces when the stock is running low.

In some embodiments, the apparatus 100 includes a track 140 coupled to the sampling device 120, wherein the sampling device 120 is moveable along the track 140 to adjust a height of the nozzle 122 relative to the user. For example as shown in FIG. 2A, the sampling device 120 is coupled to a sampling device holder 142 that is coupled to and moveable along the track 140. The user may place the hygiene mouthpiece over the nozzle 122 and manually adjust the height of the nozzle 122 by moving the nozzle 122 along the track 140, so that the nozzle 122 can be positioned near the user's mouth.

In some embodiments, the sampling device 120 is automatically adjustable based on a height of the user that is determinable from the image data of the user. More specifically, the imaging devices 112 capture the user image data which can be used to determine the user's height and position of the user's mouth. The sampling device 120 is configured to automatically move along the track 140 based on the user's height to adjust the height of the nozzle 122 relative to the user, so that the nozzle 122 is automatically adjusted to the position of the user's mouth. The nozzle 122 can thus be automatically positioned according to the user's height without the user having to touch the nozzle 122 or any part of the sampling device 120, thereby avoiding contamination of the nozzle 122 or any part of the sampling device 120.

Further as shown in FIGS. 2A and 2B, the apparatus 100 includes an analysis device 200 for receiving the breath sample from the sampling device 120 and for analysing the breath sample. For example, the analysis device 200 is disposed inside the housing body 102. For example, the apparatus 100 includes a set of internal fluidic conduits 150 fluidically connected to the sampling device 120 and the analysis device 200, the internal fluidic conduits 150 for communicating the breath sample from the sampling device 120 to the analysis device 200. The analysis device 200 may include a set of rollers 210 for rolling the analysis device 200 in and out of the housing body 102.

The analysis device 200 includes a mass spectrometer 220 for measuring a number of biomarkers in the breath sample. The analysis device 200 further includes a computer device 230 for receiving the user identification data from the input devices and for generating breath analysis results for the user based on the biomarker measurements. The breath analysis results can thus be associated with a unique user based on the user identification data.

In some embodiments, the apparatus 100 includes a heating device 160 for heating any residual breath sample in the mass spectrometer 220 and/or the sampling device 120 after measuring the biomarkers. The heating device 160 may additionally heat any residual breath sample in the internal fluidic conduits 150. The heating device 160 heats any residual breath sample such that it is maintained at a suitable temperature that kills pathogens in the residual breath sample. For example, the residual breath sample is heated to a temperature of about 70° C. to kill bacteria and viruses. The heating device 160 thus performs heat sterilisation so that the apparatus 100 remains clean and safe for the next user to use.

In some embodiments, the apparatus 100 includes a vacuum pump for evacuating any residual breath sample in the mass spectrometer 220 and/or the sampling device 120 after measuring the biomarkers. The vacuum pump may additionally evacuate any residual breath sample in the internal fluidic conduits 150. The vacuum pump may evacuate the residual breath sample to a container fluidically connected to the analysis device 200. The vacuum pump thus removes any residual breath sample of the user so that when the next user uses the apparatus 100, the breath sample from the next user does not get contaminated by the residual breath sample of the previous user.

In some embodiments, the apparatus 100 includes an exhaust fan 170 for ventilating the apparatus 100. The exhaust fan 170 may ventilate any residual breath sample to the environment and remove heated air in the housing body 102. For example, air in the housing body 102 may be heated by the heating device 160 during the heat sterilisation and this heated air is then ventilated to the environment via the exhaust fan 170. The analysis device 200 may include a set of fans for local ventilation of the mass spectrometer 220 and/or the computer device 230 to mitigate risk of overheating.

In some embodiments, the exhaust fan 170 includes a filter 172, such as a HEPA filter, for ventilating the apparatus 100 through the filter 172. The filter 172 may filter out pathogens, such as bacteria and viruses, in the residual breath sample so that the pathogens are not released to the environment. The filter 172 would be replaced regularly according to the manufacturer's guidelines.

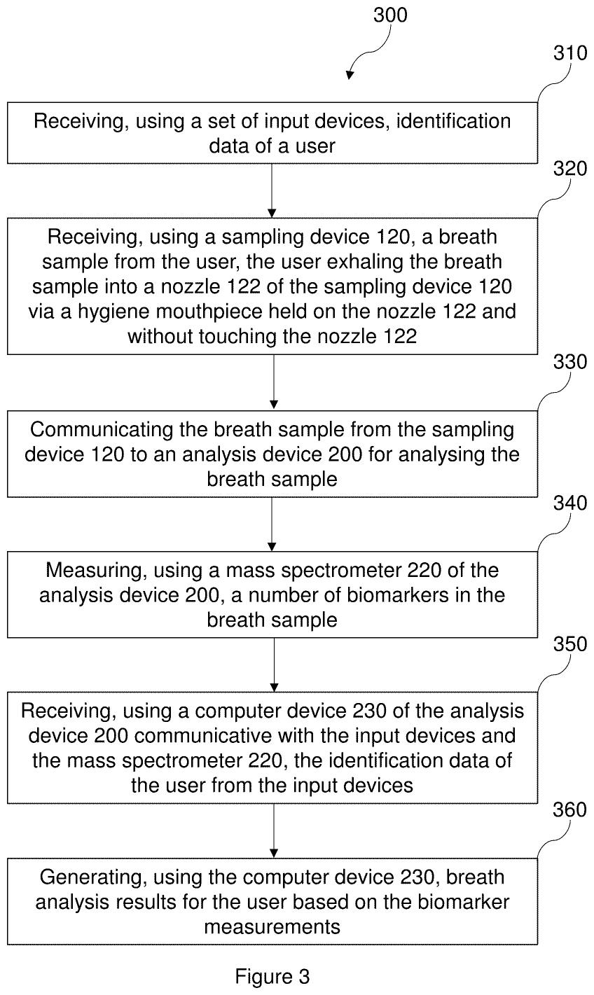

Representative or exemplary embodiments of the present disclosure also describe an automated breath analysis method 300, such as performed by the apparatus 100. With reference to FIG. 3, the method 300 includes a step 310 of receiving, using a set of input devices, identification data of a user. The method 300 includes a step 320 of receiving, using a sampling device 120, a breath sample from the user, the user exhaling the breath sample into a nozzle 122 of the sampling device 120 via a hygiene mouthpiece held on the nozzle 122 and without touching the nozzle 122.

The method 300 includes a step 330 of communicating the breath sample from the sampling device 120 to an analysis device 200 for analysing the breath sample. The method 300 includes a step 340 of measuring, using a mass spectrometer 220 of the analysis device 200, a number of biomarkers in the breath sample. The method 300 includes a step 350 of receiving, using a computer device 230 of the analysis device 200 communicative with the input devices and the mass spectrometer 220, the identification data of the user from the input devices. The method 300 includes a step 360 of generating, using the computer device 230, breath analysis results for the user based on the biomarker measurements.

In some embodiments, the apparatus 100 includes a display device 180 for displaying the breath analysis results. The display device 180 may be configured for displaying visual instructions to use the apparatus 100. The apparatus 100 may further include comprising a set of speakers 182 for generating audio instructions to use the apparatus. Instructions on how to use the apparatus 100 to perform breath analysis can be delivered through the display device 180 and speakers 182 to the user. The apparatus 100 may include a microphone 184 for the user to communicate with a remote operator in case of difficulties or problems in using the apparatus 100. The imaging devices 112 may be configured to record the whole process of the user using the apparatus 100.

In some embodiments, the computer device 230 is communicatively connected to the display device 180, speakers 182, and microphone 184. The computer device 230 is pre-installed with the instructions on how to use the apparatus 100 and sends the instructions to the display device 180 and speakers 182. The computer device 230 may optionally be configured to email and/or print the breath analysis results automatically and/or in response to a user input.

In some embodiments, the apparatus 100 may include a second computer device 190 that is communicatively connected to the display device 180, speakers 182, and microphone 184. The second computer device 190 may be placed inside the housing body 102. The second computer device 190 is pre-installed with the instructions on how to use the apparatus 100 and sends the instructions to the display device 180 and speakers 182. The second computer device 190 may optionally be configured to email and/or print the breath analysis results automatically and/or in response to a user input.

As described above, the mass spectrometer 220 is configured for measuring a number of biomarkers in the breath sample. The biomarkers may include chemical compounds such as volatile organic compounds (VOCs). Mass spectrometry is an analytical technique that is used to measure the mass-to-charge ratio of the chemical compounds in the breath sample in order to identify and quantify the chemical compounds and determine the structure and chemical properties of the chemical compounds. The mass spectrometer 220 may operate using various types of mass spectrometry, such as but not limited to, gas chromatography mass spectrometry, proton-transfer-reaction mass spectrometry, selected ion flow tube mass spectrometry, and Raman spectrometry.

The computer device 230 generates the breath analysis results based on the biomarker measurements. As mentioned above, the analysis of components present in exhaled human breath allows for monitoring of human health. More specifically, the breath analysis results, which may include the identification and quantification of biomarkers such as VOCs in the exhaled breath sample, can indicate a variety of diseases and metabolic activities and make the detection of diseases easier.

In some embodiments, the computer device 230 is configured for analysing the biomarker measurements and thereby generate the breath analysis results for the user. In some embodiments, the computer device 230 cooperates with a remote server across a communication network to generate the breath analysis results. More specifically, the computer device 230 sends the user identification data and biomarker measurements to the remote server. The remote server analyses the biomarker measurements and sends the breath analysis results to the computer device 230. The computer device 230 receives the breath analysis results from the remote server and thereby generate the breath analysis results for the user. Additionally, troubleshooting of problems with the apparatus 100 can be done remotely from the remote server without having an operator on-site at the apparatus 100.

The communication network is a medium or environment through which content, notifications, and/or messages are communicated among various components. Suitable security protocols, such as encryption protocols, may be implemented in the communication network for secure communications among the components. Some non-limiting examples of the communication network include a virtual private network (VPN), wireless fidelity (Wi-Fi) network, light fidelity (Li-Fi) network, local area network (LAN), wide area network (WAN), metropolitan area network (MAN), satellite network, Internet, fibre optic network, coaxial cable network, infrared (IR) network, radio frequency (RF) network, and any combination thereof. Various components in the communication network may connect to the communication network in accordance with various wired and wireless communication protocols, such as Transmission Control Protocol/Internet Protocol (TCP/IP), User Datagram Protocol (UDP), 2nd to 5th Generation (2G to 5G) communication protocols, Long Term Evolution (LTE) communication protocols, and any combination thereof. Each component to the communication network includes a data communication or transceiver module to communicate and transmit/receive data over the communication network. Some non-limiting examples of a transceiver module include an antenna module, a radio frequency transceiver module, a wireless transceiver module, a Bluetooth transceiver module, an Ethernet port, a Universal Serial Bus (USB) port, or any other module/component/device configured for transmitting and receiving data.

The computer device 230 may be communicatively connected directly to the remote server across the communication network. Alternatively, the computer device 230 may be communicatively connected to the second computer device 190 that is in turn communicatively connected to the remote server across the communication network. The computer devices 190,230 may be communicatively connected to each other via various wired/wireless communication protocols. The computer device 230 may send the user identification data and biomarker measurements to the remote server via the second computer device 190, and receive the breath analysis results from the remote server via the second computer device 190.

Each computer device 190,230 includes at least one processor and various other modules or components configured for performing various operations or steps. Such operations or steps are performed in response to non-transitory instructions operative or executed by the processor. The non-transitory instructions are stored on a memory of the computer device 190,230 and may be referred to as computer-readable storage media and/or non-transitory computer-readable media. Non-transitory computer-readable media include all computer-readable media, with the sole exception being a transitory propagating signal per se. Some non-limiting examples of the computer device 190,230 include computers, laptops, mini-computers, mainframe computers, any non-transient and tangible machines that can execute a machine-Readable code.

The apparatus 100 and method 300 provide an improved and automated way to analyse exhaled breath samples to allow for medical diagnosis and detection of diseases in users. Users can use the apparatus 100, which can be located at public areas, by themselves without intervention by an operator or healthcare professional. The whole breath analysis process can be performed in an automated manner that reduces operational costs, improves productivity and efficiency, and provides better user experience.

To illustrate an example of disease detection from exhaled breath analysis, when a person has respiratory disease and is infected with a virus such as coronavirus, viral shedding can occur when the person coughs or sneezes. The viral shedding can be atomized in exhaled breath which can potentially infect other people when they breathe in contaminated air. The apparatus 100 and method 300 provides an automated and non-invasive way of detecting the virus and diagnosing respiratory disease, allowing for quick screening of infected people. Early diagnosis of respiratory disease and isolation of positive cases are important strategies to curb the spread of infection.

In the foregoing detailed description, embodiments of the present disclosure in relation to an automated breath analysis apparatus and method are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. An automated breath analysis apparatus comprising:
a set of input devices for receiving identification data of a user;
a sampling device for receiving a breath sample from the user, the sampling device comprising a nozzle for the user to exhale the breath sample into the sampling device, the nozzle arranged to hold a hygiene mouthpiece such that the user exhales the breath sample via the hygiene mouthpiece without touching the nozzle; and
an analysis device for receiving the breath sample from the sampling device and for analysing the breath sample, the analysis device comprising:
a mass spectrometer for measuring a number of biomarkers in the breath sample; and
a computer device for receiving the user identification data from the input devices and for generating breath analysis results for the user based on the biomarker measurements.

2. The apparatus according to claim 1, further comprising a track coupled to the sampling device, wherein the sampling device is moveable along the track to adjust a height of the nozzle relative to the user.

3. The apparatus according to claim 2, wherein the sampling device is configured to automatically move along the track based on a height of the user to adjust the height of the nozzle relative to the user.

4. The apparatus according to claim 1, wherein the input devices comprise one or more scanning devices for scanning an identification document of the user to thereby receive the user identification data.

5. The apparatus according to claim 1, wherein the input devices comprise one or more imaging devices for capturing image data of the user.

6. (canceled)

7. The apparatus according to claim 5, wherein the sampling device is automatically adjustable to adjust the height of the nozzle relative to the user based on a height of the user that is determinable from the image data of the user.

8. The apparatus according to claim 1, further comprising a dispenser for storing a plurality of hygiene mouthpieces and for dispensing the hygiene mouthpiece to the user.

9. (canceled)

10. The apparatus according to claim 1, further comprising a heating device for heating any residual breath sample in the mass spectrometer and/or the sampling device after measuring the biomarkers.

11. The apparatus according to claim 1, further comprising an exhaust fan for ventilating the apparatus.

12. (canceled)

13. The apparatus according to claim 1, further comprising a vacuum pump for evacuating any residual breath sample in the mass spectrometer and/or the sampling device after measuring the biomarkers.

14. The apparatus according to claim 1, wherein the computer device is configured for analysing the biomarker measurements and thereby generate the breath analysis results for the user.

15. (canceled)

16. An automated breath analysis method comprising:
receiving, using a set of input devices, identification data of a user;
receiving, using a sampling device, a breath sample from the user, the user exhaling the breath sample into a nozzle of the sampling device via a hygiene mouthpiece held on the nozzle and without touching the nozzle;
communicating the breath sample from the sampling device to an analysis device for analysing the breath sample;
measuring, using a mass spectrometer of the analysis device, a number of biomarkers in the breath sample;
receiving, using a computer device of the analysis device communicative with the input devices and the mass spectrometer, the identification data of the user from the input devices; and
generating, using the computer device, breath analysis results for the user based on the biomarker measurements.

17. The method according to claim 16, further comprising automatically moving the sampling device along a track based on a track based on a height of the user to adjust the height of the nozzle relative to the user.

18. The method according to claim 16, wherein receiving the user identification data comprises scanning, using one or more scanning devices, an identification document of the user.

19. The method according to claim 16, wherein receiving the user identification data comprises capturing, using one or more imaging devices, image data of the user.

20. (canceled)

21. The method according to claim 19, further comprising automatically moving the sampling device based on a height of the user to adjust the height of the nozzle relative to the user based on a height of the user that is determinable from the image data of the user.

22. The method according to claim 16, further comprising dispensing, from a dispenser storing a plurality of hygiene mouthpieces, the hygiene mouthpiece to the user.

23. (canceled)

24. The method according to claim 16, further comprising heating, using a heating device, any residual breath sample in the mass spectrometer and/or the sampling device after measuring the biomarkers.

25-26. (canceled)

27. The method according to claim 16, further comprising evacuating, using a vacuum pump, any residual breath sample in the mass spectrometer and/or the sampling device after measuring the biomarkers.

28. (canceled)

29. The method according to claim 16, further comprising:
sending, from the computer device, the user identification data and biomarker measurements to a remote server for the remote server to analyse the biomarker measurements; and
receiving, by the computer device, the breath analysis results from the remote server and thereby generate the breath analysis results for the user.
